(19) United States
(12) Patent Application Publication
(10) Pub. No.: US 2020/0219585 A1
(43) Pub. Date: Jul. 9, 2020

(54) USING AN ESTIMATED RISK ASSESSMENT WITH CREDIBLE INTERVAL

(71) Applicant: United States of America as Represented by The Secretary of The Army, Alexandria, VA (US)

(72) Inventor: Lyle D. Burgoon, Apex, NC (US)

(21) Appl. No.: 16/240,634

(22) Filed: Jan. 4, 2019

(51) Int. Cl. G16B 15/30; G16H 50/30

(52) U.S. Cl. CPC G16B 15/30; A61B 5/7275; G16H 50/30

(57) ABSTRACT

Disclosed is an estimated risk assessment prepared for a data-poor query chemical. When searching for appropriate chemical surrogates for the read-across, rather than focusing purely on chemical structural similarities, consideration of biochemical similarities allows surrogates to be compared on how similarly they perform in the biological functions where toxicity ultimately occurs. The estimated risk assessment includes a “credible interval” determined by the confidence that the surrogacy information included in the read-across adequately mimics the as-yet-undetermined information for the query chemical. A health-hazard risk is calculated based on the credible interval and is used to set estimated potential toxicity and safe values for the query chemical.

### CROSS-REFERENCE TO RELATED APPLICATIONS

The present application is related to U.S. Patent Application (Attorney Docket Number COE-772A), which is incorporated herein in its entirety by reference.

### FEDERALLY SPONSORED RESEARCH OR DEVELOPMENT

Under paragraph 1(a) of Executive Order 10096, the conditions under which this invention was made entitle the Government of the United States, as represented by the Secretary of the Army, to an undivided interest therein on any patent granted thereon by the United States. This and related patents are available for licensing to qualified licensees.

### BACKGROUND

### Field of the Invention

The present disclosure is related generally to chemical risk assessments and, more particularly, to using data-rich surrogates to supplement information for a query chemical.

### Description of the Related Art

This section introduces aspects that may help facilitate a better understanding of the invention. Accordingly, the statements of this section are to be read in this light and are not to be understood as admissions about what is prior art or what is not prior art.

It is very expensive and time consuming to run the full battery of safety tests that are required to produce a detailed risk assessment for a new chemical. Instead, in some instances, a company can attempt to estimate risk for a new (“data-poor”) chemical by using the detailed risk assessments and other knowledge already produced for other similar and approved (“data-rich”) chemicals. Also, regulatory and emergency response agencies sometimes use information on data-rich chemicals to inform them of the potential toxicity of data-poor chemicals found at polluted sites. This risk-estimation practice is called “read-across,” and it focuses on the use of chemical similarity to identify data-rich surrogate chemicals to fill in the data gaps for data-poor chemicals.

Today, such chemical surrogacy is based on structural similarity between the “query chemical” and its potential surrogates. This surrogacy analysis is based on the idea that chemicals that have similar structures should have similar abilities to bind biological receptors on proteins and thus should cause similar toxicity.

### BRIEF SUMMARY

An estimated risk assessment is prepared for a data-poor query chemical. When searching for appropriate chemical surrogates for the read-across, rather than focusing purely on chemical structural similarities, consideration of biochemical similarities allows surrogates to be compared on how similarly they perform in the biological functions where toxicity ultimately occurs. The estimated risk assessment that is produced includes a “credible interval” determined by the confidence that the surrogacy information included in the read-across adequately mimics the as-yet-undetermined information for the query chemical. A health-hazard risk is calculated based on the credible interval and is used to set estimated potential toxicity and safe values for the query chemical.

### BRIEF DESCRIPTION OF THE SEVERAL VIEWS OF THE DRAWINGS

While the appended claims set forth the features of the present techniques with particularity, these techniques, together with their objects and advantages, may be best understood from the following detailed description taken in conjunction with the accompanying drawings of which:

FIG. 1 is a generalized overview of a method for creating an estimated risk assessment;

FIG. 2 is a structural depiction of an exemplary query chemical, 17alpha-ethinylestradiol;

FIG. 3 is a structural depiction of an exemplary surrogacy candidate, 17beta-estradiol;

FIG. 4 is a flowchart of a method for producing an estimated risk assessment;

FIG. 5 is a flowchart of a method for choosing among surrogacy candidates and, as such, is an expansion of step 404 of FIG. 4;

FIG. 6 is a schematic of an exemplary system for performing the methods of FIGS. 4 and 5;

FIG. 7 is a flowchart of a method for using an estimated risk assessment; and

FIG. 8 is flowchart for generating a suggestion to perform a toxicological study.

### DETAILED DESCRIPTION

Detailed illustrative embodiments of the present invention are disclosed herein. However, specific structural and functional details disclosed herein are merely representative for purposes of describing example embodiments of the present invention. The present invention may be embodied in many alternate forms and should not be construed as limited to only the embodiments set forth herein. Further, the terminology used herein is for the purpose of describing particular embodiments only and is not intended to be limiting of example embodiments of the invention.

As used herein, the singular forms “a,” “an,” and “the” are intended to include the plural forms as well, unless the context clearly indicates otherwise. It further will be understood that the terms “comprises,” “comprising,” “includes,” and “including” specify the presence of stated features, steps, or components but do not preclude the presence or addition of one or more other features, steps, or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

Although it is often true that chemicals that look structurally similar have similar toxicity, it is not always true. There could be subtleties in how a particular chemical binds to a particular protein that cause the chemical's toxicity to differ from the toxicity of structurally similar chemicals. This is the case, for example, for the selective estrogen receptor modulators.

Another issue is just how similar is similar when it comes to chemical structure? Chemical families are composed of chemicals that share similar chemistries. However, their toxicities can differ depending upon how structurally flexible certain members of the family are when compared to others. There are also the well known examples of different stereoisomers of chemicals that have radically different biological activities—this is a case of chemicals that look largely the same having very different activity.

The present disclosure teaches a method for predicting or identifying the hazards of a chemical, the likely dose-response relationship for a chemical, the environmental fate and transport of a chemical, and a toxicity reference value (e.g., reference dose (“RfD”), reference concentration (“RfC”)) that is likely to protect either a human or animal population. Further, by combining all of the previously mentioned information, a site-specific estimated risk assessment can be produced.

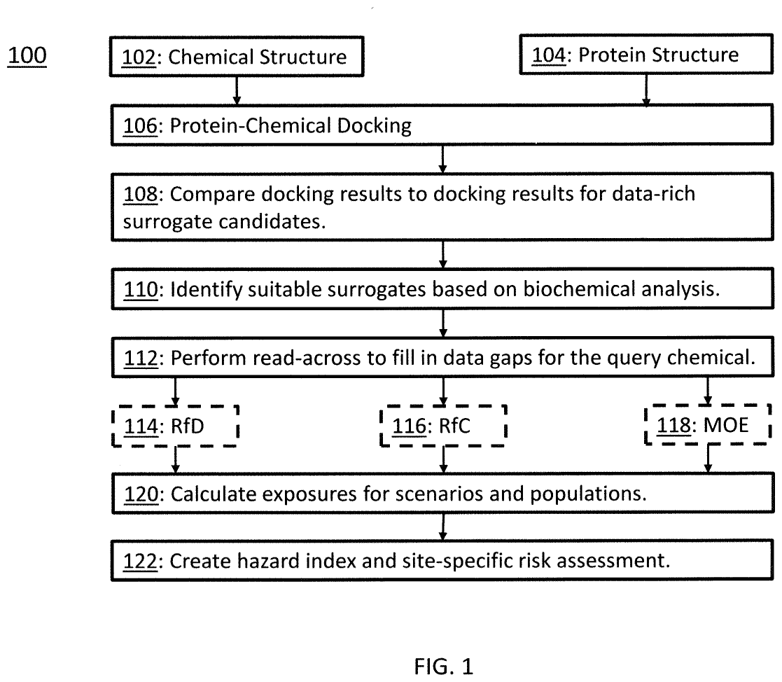

FIG. 1 is an exemplary overview of a method 100 for generating an estimated risk assessment based upon a chemical surrogate, or upon multiple chemical surrogates, for a given query chemical. The method identifies likely chemical surrogates by using a chemical's likely biochemical interactions with a protein of interest. A “good” chemical surrogate is one whose binding pattern to the protein is similar to that of the query chemical. A similar binding pattern means that the surrogate chemical has interaction points with the protein that are highly similar to the interaction points of the query chemical with the protein. Here, interaction points are those places where a chemical is likely to be interacting with the protein. Interaction occurs when non-covalent (or non-electrical) bonds are formed, such as hydrogen bonds and van der Waals forces.

This definition of a “good” chemical surrogate, wherein the surrogate's interactions with the protein of interest are highly similar to those of the query chemical, is sufficient to infer that the chemical surrogate is interacting with the protein in the same way as the query chemical does and is thus taking up the same space. This is, in turn, sufficient to infer that the surrogate chemical and the query chemical are likely to induce the same biochemical changes in the protein and result in the same biological outcomes.

Surrogate chemicals should have more toxicological, or at least different toxicological, data than the query chemical, so that data gaps associated with the query chemical can be filled with information from the surrogate chemicals. This type of data-gap filling is known as “read-across” and is a method used by chemical companies and government regulatory agencies around the world. It helps risk assessors at companies and governments to make judgments about chemicals when important information is lacking.

The method 100 of FIG. 1 begins by reading in chemical structural information for the query and surrogate chemicals 102 as well as for the protein of interest that these will be docked into 104. The protein-chemical docking occurs virtually in a system 106 that analytically performs the docking. The docking system 106 produces files that list the affinities of the query and surrogate chemicals for the protein and the best locations where the query and surrogate chemicals are likely to sit within the protein. Whenever possible, the pose of the query chemical within the protein is checked against a known chemical bound to the protein to ensure that the best pose of the query chemical is identified.

The chemical contact points of the query chemical with the protein are then compared 108 to the contact points for the candidate surrogate chemicals. Candidate surrogate chemicals with the “best” similarity are identified as the surrogate chemicals 110. Contact-point similarity can be calculated in many ways, but the most straightforward way is to calculate the Jaccard Index, whereby the sizes (i.e., the number of amino acids) of the intersection of the amino acids being contacted in the query and candidate surrogate chemicals are divided by the size (number of amino acids) of the union of the amino acids being contacted by the query and candidate surrogate chemicals.

The Bayesian read-across approach 112 is used to fill data gaps for the query chemical. Where multiple surrogate chemicals could fill the same data gap, the Bayesian bootstrap approach can be used, with weighting on the sampling based on the Jaccard Index. That is, if the Jaccard Index is 40% for surrogate chemical A and 90% for surrogate chemical B, then surrogate chemical A represents 0.4/(0.4+0.9)=31% of the weighting, and surrogate chemical B represents 0.9/(0.4+0.9)=69% of the weighting. The missing data point for the query chemical is the mean or the median of the distribution from the Bayesian bootstrap, and the uncertainty is generally set to be the centered 90%, 95%, or 99% of the distribution, also known as the 90%, 95%, or 99% credible interval.

This information can be used to derive an RfD 114, RfC 116, or margin of exposure level (“MOE”) 118. In some cases, some of these values 114, 116, 118 are already known for one or more of the surrogate chemicals and can be read into the estimated risk assessment. The RfD 114, RfC 116, or MOE 118 is then used to estimate risk-assessment values that can be used at specific sites to determine the risk posed by the query chemical 120, 122.

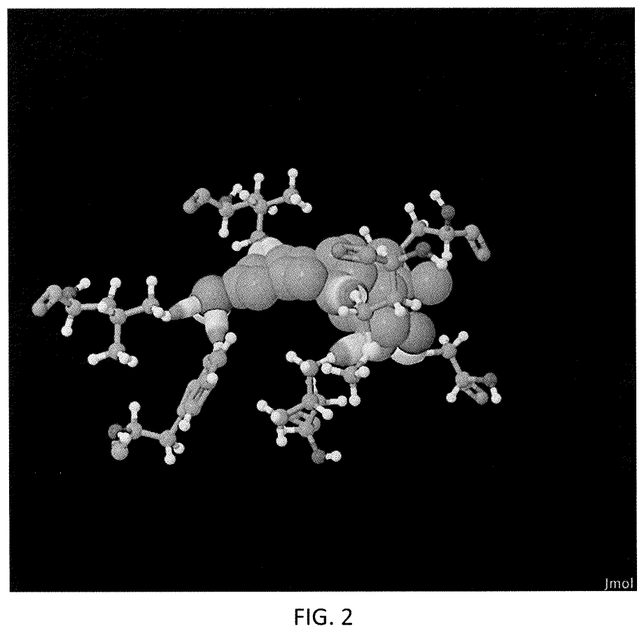

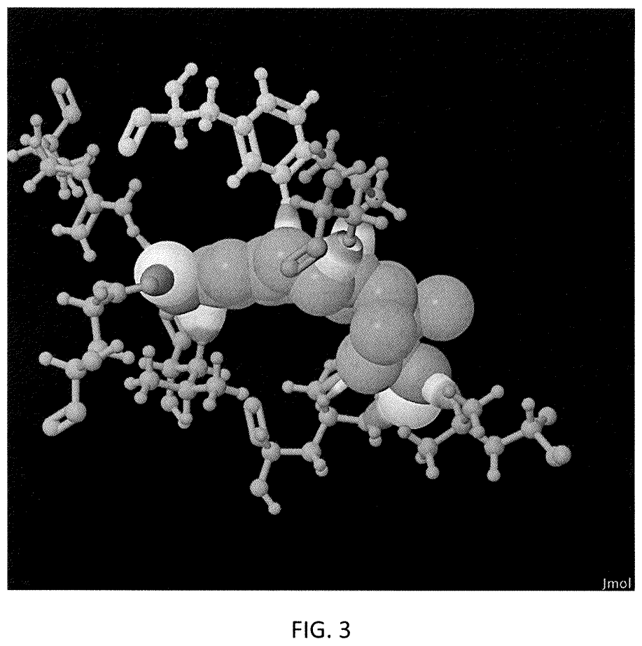

FIGS. 2 and 3 show examples of the docking and points of contact of a query chemical, 17alpha-ethinylestradiol (FIG. 2), and a candidate surrogate chemical, 17beta-estradiol (FIG. 3). These figures were produced by software that takes in the output from docking programs and visualizes the docking output. The query chemical exhibits contact with 7 amino acids, while the candidate surrogate chemical exhibits contact with 6 amino acids. All 6 of the amino acids that the candidate surrogate contacts are also in the set of 7 amino acids that the query chemical contacts. This results in a Jaccard Index of 6/7=86%. It should be noted that in this example the pose, or where the candidate surrogate sits in the protein of interest, is not the most optimal pose found by the docking software. However, this is the pose that results in the highest Jaccard Index with the query chemical, and the query chemical is sitting in the best spot within the protein-binding pocket for known ligands (i.e., chemicals that bind to the protein). Thus, 17beta-estradiol (FIG. 3) is a high quality surrogate chemical for 17alpha-ethinylestradiol (FIG. 2).

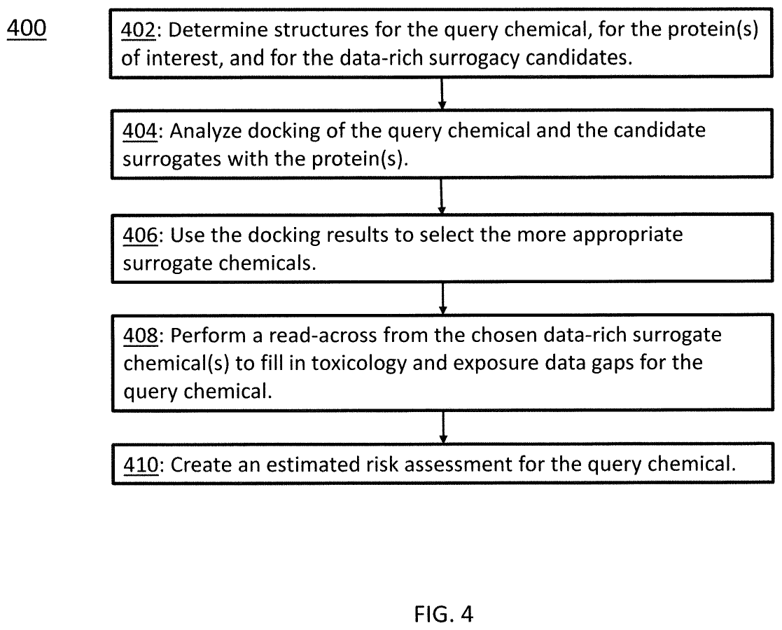

FIG. 4 is a flowchart of a method 400 for generating an estimated chemical risk assessment for a query chemical based upon identification of one or more surrogate chemicals. In step 402, structural information for the query chemical, for the protein of interest, and for candidate surrogate chemicals is received. Candidate surrogate chemicals may have already been screened previously. In fact, it would be desirable to have an existing database of previously screened candidate surrogate chemicals for many protein receptors of interest.

Step 404 takes the structural information from step 402 and performs protein-chemical docking for the query chemical and for all candidate surrogate chemicals against the protein structure. In other words, a docking software algorithm examines the best poses (where a chemical sits in an X, Y, Z three-dimensional coordinate system) with respect to the protein's binding site.

Step 406 determines the better surrogate chemicals based on the similarity of the poses for each surrogate chemical when compared to the query chemical. The most similar surrogate chemicals are chosen to move into step 408.

In step 408, a read-across is performed using known data from the chosen surrogate chemicals to fill in toxicology and exposure data gaps possibly including reference toxicity values and MOE values. The read-across process in step 408 can take many different forms. Read-across is typically performed by identifying data gaps for toxicology and exposure information for the query chemical. It is not unusual for a query chemical to have no data. Next, data gaps for the query chemical are filled using either the lowest (most health conservative) value available across all of the chosen surrogate chemicals or the mean or median of all the values across multiple surrogate chemicals. For instance, if the query chemical is missing a human oral RfD, then the RfDs for all surrogate chemicals are identified, if available. The read-across RfD in the estimated risk assessment produced for the query chemical may then be one of: 1) the lowest RfD across all of the surrogate chemicals, 2) the median RfD across all of the surrogate chemicals, or 3) the mean RfD across all of the surrogate chemicals. This process could be used for other toxicological (human or ecological health) endpoints and for exposure endpoints (e.g., environmental fate and transport factors, equations, or the like). This approach works for all exposure and toxicological values, including other regulatory values such as LD50 (the dose that causes 50% death in the population), LC50 (the concentration that causes 50% death in the population), and physical-chemical properties that determine likely exposure levels. The read-across in step 408 is also used to identify potential health hazards for hazard assessment. Similar to identifying quantitative information, the potential health hazards are lists of health hazards that apply for any of the chosen surrogate chemicals.

In step 410, the reference toxicity values or MOE values from step 408 are used to generate an estimated risk assessment for the query chemical for the particular endpoint associated with the protein of interest. The estimated risk assessment generated at step 410 follows standard chemical risk-assessment practices. These include a hazard assessment to identify potential hazards, a dose-response analysis to identify a point of departure that is transformed into a safe exposure value, and an exposure assessment that identifies how much of the query chemical a human or animal is likely to be exposed to. All of these values are obtained from the read-across process in step 408. A human or ecological health risk is said to exist at a particular site if the read-across exposure levels, or the actual exposure levels (if the query chemical can be measured at the site), is above the safe level determined in step 408.

In some embodiments, the estimated risk assessment generated in step 410 includes surrogacy information to inform users that this assessment is partly based on surrogacy information. The estimated risk assessment may also include information about the calculated credible intervals (see the above discussion of step 112 of FIG. 1).

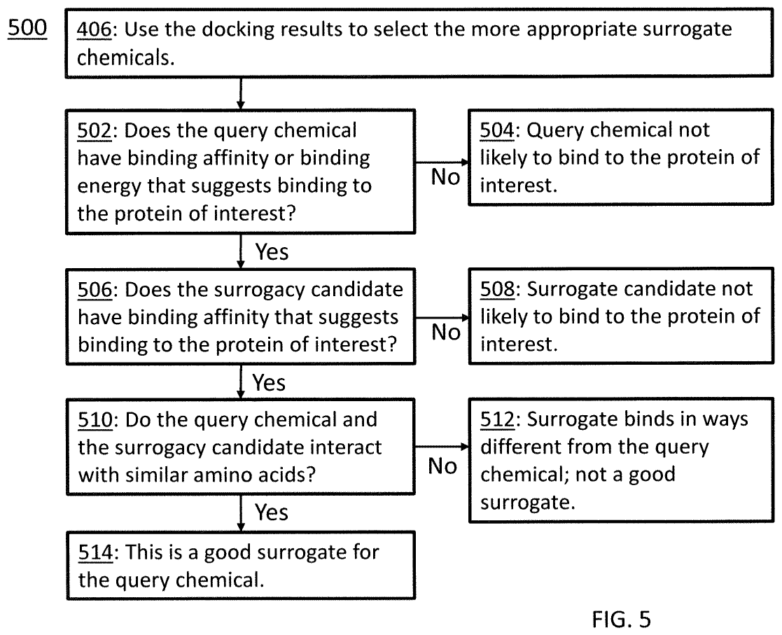

FIG. 5 is a flowchart of an exemplary method 500 for determining the best surrogates based on similarity of poses between the query chemical and potential surrogate chemicals. This method 500 explains in further detail the process that underlies step 406 of FIG. 4. Step 502 receives the output from the protein-chemical docking process and asks the question whether or not the query chemical has a binding energy that suggests that the query chemical binds to the protein of interest. Because industrial chemicals are not designed to bind to proteins, it is not uncommon to generally err on the side of being health protective. In other words, chemical affinity values in the 1 to 100 millimolar range can still qualify as an energy that suggests binding to the protein. This threshold value is user-defined. For example, if the threshold is set at 100 millimolar, then if the binding affinity is greater than 100 millimolar, the answer to the question in step 502 is No, and the process enters step 504. The query chemical is not likely to bind to the protein, the process 500 in FIG. 5 ends, and the process 400 in FIG. 4 ends.

If, on the other hand, the binding affinity is less than the user-defined threshold, then the answer to the question posed in step 502 is Yes. The process 500 proceeds to step 506 which asks if the potential surrogate chemical also has a binding affinity that suggests binding to the protein. The same user-defined threshold is used in the same manner as in step 502. If the potential surrogate chemical has an affinity greater than the user-defined threshold, then the process proceeds to step 508, where this potential surrogate chemical is declared to not bind the protein, and the process 500 ends by discarding this particular surrogate chemical.

If the binding affinity is less than the user-defined threshold at step 506 (the answer is “Yes”), then the surrogate chemical is likely to bind the protein, and the process 500 proceeds to step 510. Step 510 seeks to identify if the pose of the surrogate chemical sitting in the protein is the same or similar to the pose of the query chemical when it also sits in the protein. From a biochemical standpoint, the contact/interaction points between the protein and the surrogate chemical and between the protein and the query chemical are most important. Because the protein and the chemicals are three-dimensional, and because some chemicals have rotation points where they can be rotated around, it is the interaction of a chemical with specific amino acids in the protein that is most important in determining how that chemical will impact the protein's activity. In other words, two chemicals can both occupy the binding site of a protein, but what determines whether the two chemicals are likely to activate or to inhibit the protein in the same way and to the same degree is the combination of amino acids that the chemical is interacting with or coming into contact with. For instance, one chemical could be shaped slightly differently from another but occupy largely the same space in the protein. If both chemicals are not interacting with the same amino acids, then the protein may exhibit different behavior, such as a slightly different movement of a large protein helix, which may confer different activity. This is how selective estrogen-receptor modulators are believed to act: Chemicals with somewhat similar shapes can have dramatically different estrogen-receptor activities in the same tissues. This can manifest as the same chemical acting as a weak estrogen in one tissue, a strong estrogen in another, while blocking all estrogenic activity in yet another tissue. Tamoxifen is a good example of this, especially when compared to an endogenous estrogen such as 17-beta-estradiol.

In step 510, there are many ways to measure “similarity” with respect to the amino acids being contacted or interacted with. One measure, the Jaccard Index, is illustrated in the above discussion of FIGS. 2 and 3. There are versions of the Tanimoto similarity that are mathematically identical to the Jaccard Index. In addition, the simple matching coefficient (“SMC”) is also mathematically identical to the Jaccard Index in this usage. Specifically, the SMC also provides for a term that measures absence in both sets; because that term does not matter here, it cancels out, and the SMC calculation simplifies to the Jaccard Index.

A user-defined threshold for the similarity is established and is the deciding criterion for whether the process 500 moves to step 512 or to step 514. If the similarity measure is less than the threshold, then the surrogate chemical is not similar enough to the query chemical, and the process 500 moves to step 512. At step 512, the potential surrogate chemical is discarded from further consideration, which ends this process 500 and the process 400 of FIG. 4. Otherwise, the similarity measure is greater than the threshold, and the process 500 moves to step 514, where the potential surrogate chemical is reclassified as a best surrogate chemical for the query chemical, and the process 500 re-enters the process 400 of FIG. 4, completing step 406.

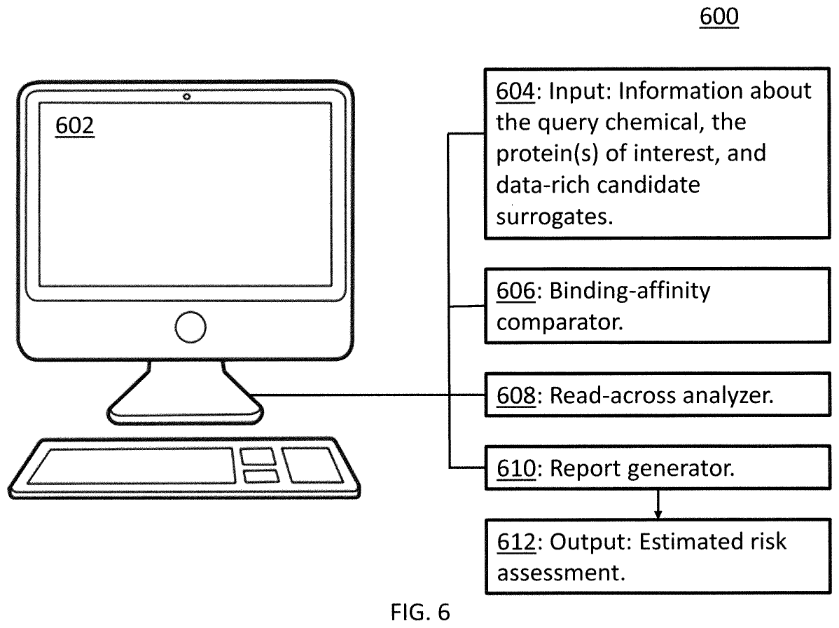

FIG. 6 is a schematic of a generalized system 600 for performing the methods discussed above. A computing device 602 is configured to access known information 604 about the query chemical, the protein of interest, and the candidate surrogate chemicals. As briefly mentioned above in reference to step 402 of FIG. 4, this information may reside in a database or in libraries hosted by chemical companies or government organizations.

The binding-affinity comparator 606 is a known software program that, using the structural information 604, calculates how and how well the query chemical and the various candidate surrogate chemicals bind to the protein of interest. In general, the binding-affinity comparator 606 performs the work of step 404 of FIG. 4.

The read-across analyzer 608 pulls information already known about the “best” data-rich surrogates and uses that information to fill in toxicity and other data gaps for the query chemical.

With the data gaps filled, the report generator 610 creates the estimated risk assessment 612 for the query chemical. The format of, and information contained within, the estimated risk assessment 612 are as expected in the industry with the possible additions of information about the surrogates used to create this assessment 612 and the credible intervals calculated based on the use of those surrogates.

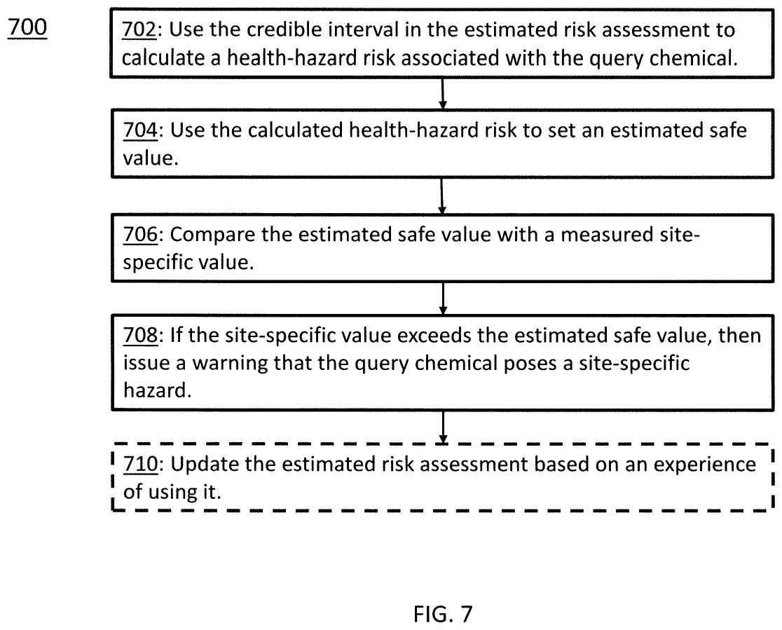

Once the estimated risk assessment 612 is generated, it can be used just like any other risk assessment as known in the art. There are cases, however, where the unique aspects of the estimated risk assessment 612 can be leveraged. Because there are many such possibilities, the method 700 of FIG. 7 is merely an overview of some of them.

The method 700 begins in step 702 where the credible interval of the estimated risk assessment is used to calculate a health-hazard risk associated with the query chemical. The health-hazard risk can be one of a hazard assessment, a dose-response assessment, an exposure assessment, a reference dose, and a reference concentration, all of which should be familiar from the discussion above. (Note that the language of step 702 is meant to be very generic: specific examples are discussed below.)

In step 704, the calculated health-hazard risk of the query chemical serves as a basis for determining an estimated safe value for use or exposure of the query chemical.

Next, in some cases the estimated safe value is compared against a value measured at a specific site (step 706). If the actual measured value exceeds the estimated safe value, then an exposure warning is issued in step 708, and remedial steps can be taken.

Procedures roughly following the above steps may be performed many times for many specific sites. Optional step 710 collects the experience of those procedures and updates the estimated risk assessment 612 as appropriate.

In one example of the method 700, the median of the credible interval in the estimated risk assessment 612 is used to calculate a most likely exposure level along with a credible interval of dose-response for that most likely exposure level. That credible interval, in turn, serves as the basis for calculating a median probability of hazard. In another example, the most likely exposure level is calculated based on a median of a credible interval of exposure. Then, a credible range of toxicity associated with the calculated most likely exposure level can be identified.

To give just one more example, a credible interval of dose-response in the estimated risk assessment 612 is used to estimate a public-health risk of toxicity associated with a set de minimis risk.

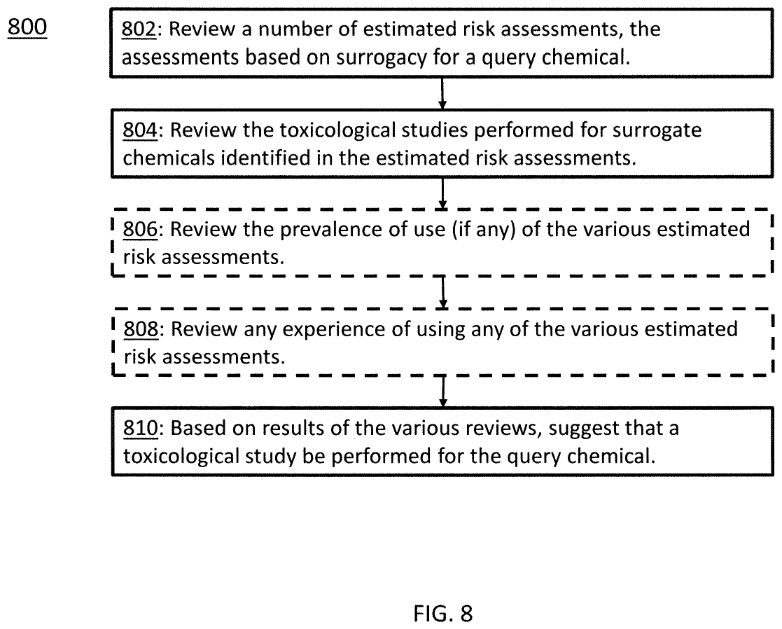

The drivers for the discussion so far are the lack of complete toxicological information for the query chemical and how to deal with that lack. Eventually it may be appropriate to simply perform the necessary (but admittedly expensive) toxicological studies to fill in the data gaps with actual information rather than continuing to rely on estimates made from chemical surrogates. The method 800 of FIG. 8 presents some guidelines for deciding when to take that step.

The method 800 begins in step 802 with reviewing any number of risk assessments 612 for the query chemical that are based on surrogacy estimates. Also reviewed, in step 804, are the underlying toxicological studies for the surrogate chemicals.

In addition to the above reviews, it may be possible and prudent to review how widespread has been the use of the estimated risk assessments 612 (step 806). Clearly, a much-used assessment 612 highlights the importance of the query chemical and may indicate that the stronger information provided by full toxicological studies may be worth the effort and expense of undertaking those studies.

Further, the real world experiences of using the estimated risk assessments 612 can be considered in step 808. When various measured results seem to be in keeping with the predictions provided by the risk assessment 612, there may be no need to do any further toxicological testing.

In step 810, all of the above considerations are weighed together to decide whether the existing estimated risk assessments 612 are performing well or whether the suggestion should be made that the estimated risk assessments 612 be replaced with traditional risk assessments based on to-be-conducted toxicological studies of the query chemical.

The techniques of the present disclosure are widely applicable beyond the discussed uses of estimating risks for chemicals introduced into foodstuffs and medicines. While no attempt is made to list all possible areas of use, a few interesting ones are noted here.

The present techniques allow new compounds in cosmetics and fragrances to be introduced without having to first perform expensive and ethically problematic animal testing. The same can be said for new materials that come into contact with foods (e.g., during industrial food manufacturing or restaurant food production).

Hydraulic fracturing (or “fracking”) is a widespread technique for increasing oil-well production, but it introduces many untested chemicals into the environment. The present techniques could be used to assess the risks and possible hazards of those introduced chemicals. Other chemicals are released into the environment or used by industry or the military and could be tested relatively cheaply. Maybe more significantly, Superfund sites and other strongly polluted locations often contain a welter of possibly dangerous chemicals. Many of those chemicals have not been tested for risk, and many have not even been identified.

In addition to producing estimated risk assessments, the present techniques can be slightly modified to identify potential off-target toxicity that would otherwise not be expected, to identify existing receptors that may mediate toxicity in some situations but that today are not considered, and to predict toxicity specific to sensitive species or to threatened or endangered populations.

The present techniques can even be used to identify unsuspected hazardous materials such as emerging or novel chemical or biological warfare agents.

Unless explicitly stated otherwise, each numerical value and range should be interpreted as being approximate as if the word “about” or “approximately” preceded the value or range.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the testing measurements.

It will be further understood that various changes in the details, materials, and arrangements of the parts which have been described and illustrated in order to explain embodiments of this invention may be made by those skilled in the art without departing from embodiments of the invention encompassed by the following claims.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

All documents mentioned herein are hereby incorporated by reference in their entirety or alternatively to provide the disclosure for which they were specifically relied upon.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

In view of the many possible embodiments to which the principles of the present discussion may be applied, it should be recognized that the embodiments described herein with respect to the drawing figures are meant to be illustrative only and should not be taken as limiting the scope of the claims. Therefore, the techniques as described herein contemplate all such embodiments as may come within the scope of the following claims and equivalents thereof.

We claim:

1. A method for using an estimated risk assessment generated for a query chemical, the estimated risk assessment comprising a credible interval, the method comprising:
first calculating an estimated health-hazard risk associated with the query chemical, the first calculating based, at least in part, on the credible interval;
using the calculated estimated health-hazard risk to set an estimated safe value;
comparing the estimated safe value with a site-specific value; and
if the site-specific value exceeds the estimated safe value, then issuing a warning that the query chemical poses a site-specific hazard.

2. The method for using an estimated risk assessment of claim 1 wherein the estimated health-hazard risk is selected from the group consisting of: a hazard assessment, a dose-response assessment, an exposure assessment, a reference dose, and a reference concentration.

3. The method for using an estimated risk assessment of claim 2 wherein the health-hazard risk comprises a hazard assessment enumerating one or more potential hazards posed by the query chemical, the potential hazards based, at least in part, on an element selected from the group consisting of: proteins that the query chemical is likely to bind to, an adverse outcome pathway, and potential hazards associated with a surrogate chemical identified by surrogacy information in the estimated risk assessment.

4. The method for using an estimated risk assessment of claim 2 wherein the health-hazard risk comprises a dose-response assessment enumerating a potential point of departure and a toxicity reference value.

5. The method for using an estimated risk assessment of claim 2 wherein the health-hazard risk comprises an exposure assessment enumerating one or more potential exposure pathways.

6. The method for using an estimated risk assessment of claim 1 further comprising:
second calculating a most likely exposure level, the second calculating based, at least in part, on a median of a credible interval of exposure; and
identifying a median probability of hazard, the identifying based, at least in part, on a credible interval of dose-response associated with the calculated most likely exposure level.

7. The method for using an estimated risk assessment of claim 1 further comprising:
second calculating a most likely exposure level, the second calculating based, at least in part, on a median of a credible interval of exposure; and
identifying a credible range of toxicity associated with the calculated most likely exposure level.

8. The method for using an estimated risk assessment of claim 1 further comprising:
second calculating an estimated public-health risk of toxicity associated with a set de minimis risk, the second calculating based, at least in part, on a credible interval of dose-response.

9. The method for using an estimated risk assessment of claim 1 further comprising:
updating the estimated risk assessment based, at least in part, on an experience of using the estimated risk assessment.

10. An estimated risk assessment for a query chemical generated by a method comprising:
identifying a first data-rich chemical surrogate for the query chemical, the identifying comprising comparing a binding affinity between the query chemical and a protein of interest with a binding affinity between the first data-rich surrogate chemical and the protein of interest;
reading-across risk-assessment values for the first data-rich surrogate chemical into the estimated risk assessment for the query chemical; and
including a credible interval in the estimated risk assessment.

11. The estimated risk assessment of claim 10 wherein the method further comprises:
identifying a second data-rich chemical surrogate for the query chemical, the identifying comprising comparing a binding of the query chemical to a protein of interest with a binding of the second data-rich surrogate chemical to the protein of interest;
wherein the reading-across comprises reading-across a combination of risk-assessment values for the first and second data-rich surrogate chemicals into the estimated risk assessment for the query chemical.

12. The estimated risk assessment of claim 11 wherein the combination of risk-assessment values is produced using a technique selected from the group consisting of: Bayesian bootstrapping, taking a lowest value, taking a mean value, taking a median value, and taking a most health-conservative value.

13. The estimated risk assessment of claim 10 further comprising:
further information based, at least in part, on the reading-across, the further information selected from the group consisting of: a hazard assessment, a dose-response assessment, an exposure assessment, a reference dose, and a reference concentration.

14. The estimated risk assessment of claim 13 wherein the further information comprises a hazard assessment enumerating one or more potential hazards posed by the query chemical, the potential hazards based, at least in part, on an element selected from the group consisting of: proteins that the query chemical is likely to bind to, an adverse outcome pathway, and potential hazards associated with the surrogate chemical.

15. The estimated risk assessment of claim 13 wherein the further information comprises a dose-response assessment enumerating a potential point of departure and a toxicity reference value.

16. The estimated risk assessment of claim 13 wherein the further information comprises an exposure assessment enumerating one or more potential exposure pathways.

17. The estimated risk assessment of claim 10 wherein the method further comprises:
updating the estimated risk assessment based, at least in part, on an experience of using the estimated risk assessment.

18. A method for generating a suggestion to perform a toxicological study for a query chemical, the method comprising:
reviewing a plurality of estimated risk assessments, each estimated risk assessment generated based on surrogacy for the query chemical, each estimated risk assessment comprising surrogacy information and a credible interval;
reviewing toxicological studies performed for data-rich surrogates identified by the surrogacy information in the plurality of estimated risk assessments; and
suggesting that a toxicological study be performed for the query chemical, the suggesting based, at least in part, on the toxicological studies performed for the data-rich surrogate chemicals and on the credible intervals in the plurality of estimated risk assessments.

19. The method for generating a suggestion to perform a toxicological study of claim 18 wherein the suggesting is further based on a prevalence of use of the plurality of estimated risk assessments.

20. The method for generating a suggestion to perform a toxicological study of claim 18 wherein the suggesting is further based on experience of using at least one of the plurality of estimated risk assessments.
